(19) United States
(12) Patent Application Publication
(10) Pub. No.: US 2002/0018851 A1
(43) Pub. Date: Feb. 14, 2002

(54) Manufacturing method for surgical implants having a layer of bioactive ceramic coating

(76) Inventors: Edward Chang, Tainan (TW); Bang-Yen Chou, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/971,733

(22) Filed: Oct. 9, 2001

Related U.S. Application Data
(63) Continuation-in-part of application No. 09/598,685, filed on Jun. 21, 2000.

(51) Int. Cl. A61L 2/00; B05D 1/36; B05D 1/08; B05D 3/00; B05D 7/00; B05D 1/02

(52) U.S. Cl. 427/299; 427/421; 427/2.1; 427/446; 427/402

(57) ABSTRACT

An improved method for the strengthening of the bond strength of a bioactive ceramic coating for a surgical implant is provided where the metallic core material is exposed to a cooling treatment prior to thermal spraying. The metallic core material, or the metallic core material with an inner ceramic layer, when more than one layer of ceramic is to be coated, is cooled to a temperature preferably in the range of −10 to 10° C. before each layer of ceramic is applied by thermally spraying on the material. By use of the cooling treatment in the method, the bond strength of the thermally-sprayed coating(s) on the surgical implant can be increased.

### RELATIONSHIP TO PRIOR APPLICATION

This Application is a Continuation-in-Part Application of Ser. No. 09/598,685, filed Jun. 21, 2001, entitled “MANUFACTURING METHOD FOR SURGICAL IMPLANTS HAVING A LAYER OF BIOACTIVE COATING”.

### BACKGROUND OF THE INVENTION

1. Field of the Invention

This invention relates to a manufacturing method for surgical implants having a layer of bioactive ceramic coating thereon. More particularly, this invention is directed to a method of manufacturing a metal implant whose surface is plated with a layer of bioactive ceramic, with the ceramic layer having good adhesion and bonding strength with the metal material.

2. Prior Art

Surgical implants such as artificial bones or artificial tooth compensate for lost functions of limbs or joints caused by illness or accidents, or the lost function of teeth caused by old age or illness.

After a doctor implanted material into bone of a human body, biological reaction produced by bone organization to the material can be classified into bioactive material, bioinert material, biotolerant material and bioresorbable material. The bioactive material is represented by hydroxyapatite, bioglass and bioglass ceramics. After this material is implanted, it has capacity of leading to bone growth, and produces chemical bonding directly with the bone organization. But if it is used independently, its strength may be poor, unable to endure a load and product a break. Next, the bioinert material is represented by aluminum oxide (Al₂O₃) (ASTM F 603-83) and zirconium oxide (ZrO₂), having the stabilized characteristic of not producing a chemical reaction to the bone organization, despite long-time contact therewith after it is implanted. A single difference is new bone organization directly making apposition on the bioinert material under proper conditions, with no fiber organization produced between them, having capabiltiy of osseointegration.

At present, metal implants of bioinert material are generally used, having a layer of bioactive ceramics plated on their surfaces, in order to reinforce affinity and combination effect between the implants and the corresponding bone organization. At the same time, the plating process is always affected by plasma spraying.

Plasma spraying is a well-known art used in plating bioactive ceramics, particularly hydroxyapatite (HA), on the metal implants. In manufacturing it, a mixed gas current is forced to pass through an electric arc, to convert the molecules of the gas and form plasma flame of high temperature. At the same time, hydroxyapatite (HA) powder is placed into this plasma flame, colliding with th surface of the metal implant by flowing with high-speed gas current, with the metal implant being cooled by dry cool air so that the molten liquid is cooled rapidly to form a coated layer on the surface of the metal.

The layer of the bioactive ceramics formed by the plasma spraying process on the surface of the metal implant gives it advantages of the metal and the bioactive ceramics. The implants thus made not only have strength of the metal to resist shock and load, but also provides the capability of osseoinduction and osseointegration by the bioactive ceramic layer having pores, permitting the bone organization to produce a strong bond in the pores of the surface of the layer, which is helpful to stabilize the implant after planted.

However, the above described art is considered to still have disadvantages, and a comparatively important one is that pores and micro gaps in the layer of the bioactive ceramics may be detrimental to the peripheral adhesion between the coated layer and the underlying metal material. Consequently, such reduces fixation of the bone organization with the coated layer. And one point worthy of further note, is that metal ions leach out of the metal material that may invade bone cells around the metal material, having a relationship to a cause of bone cancer.

In the category of prior art in the field of surgical metal material implanting, multi-layer coating comprising an inner layer and an outer layer of ceramics, has been disclosed to conquer some of the above-described disadvantages.

For example, Japanese Patent No. 5-50737 discloses a surgical implant wherein an inner layer is formed by spraying a layer of ceramics on a metal implant, and the material of the ceramics may be Al₂O₃, TiO₂, ZrO₂, or SiO₂, etc. The inner ceramic layer formed of these materials should be non-porous. Then, an outer layer of porous ceramic containing hydroxyapatite (HA) is sprayed on the inner layer.

Another Japanese Patent, No. 63-160666, discloses an improved surgical implant, where a metal layer is sprayed on a ceramic inner layer, and then an outer porous ceramic containing hydroxyapatite (HA) is finally sprayed thereon.

Next, U.S. Pat. No. 5,480,438 discloses an improved surgical implant, where an inner layer containing bioactive ceramics of more than 50 vol % glass is sprayed, and then an outer layer of porous bioactive ceramics containing less than 50 vol % glass is sprayed on.

In those case mentioned above, plural plated layers can effectively hamper ions from leaching out of the metal material, but there are still pores and micro gaps in the outer layer of plasma sprayed bioactive ceramics that may still be detrimental to the peripheral adhesion between the outer layer and the inner layer.

### SUMMARY OF THE INVENTION

The objective of the invention is to offer a manufacturing method for surgical implants having a bioactive ceramic coating layer to produce an improved surgical implant not only preventing metal material from leaching out ions, but also compensating for detriment to the peripheral adhesion between the ceramic plated layer and the inner layer of the metallic core material.

### BRIEF DESCRIPTION OF THE DRAWINGS

This invention will be better understood by referring to the accompanying drawings, wherein:

FIG. 1 is a flow chart of a first manufacturing method for surgical implants having a layer of bioactive ceramics coating of the present invention.

FIG. 2 is a flow chart of a second manufacturing method for surgical implants having a layer of bioactive ceramics coating of the present invention.

### DETAILED DESCRIPTION OF THE PREFERRED EMBODIMENTS

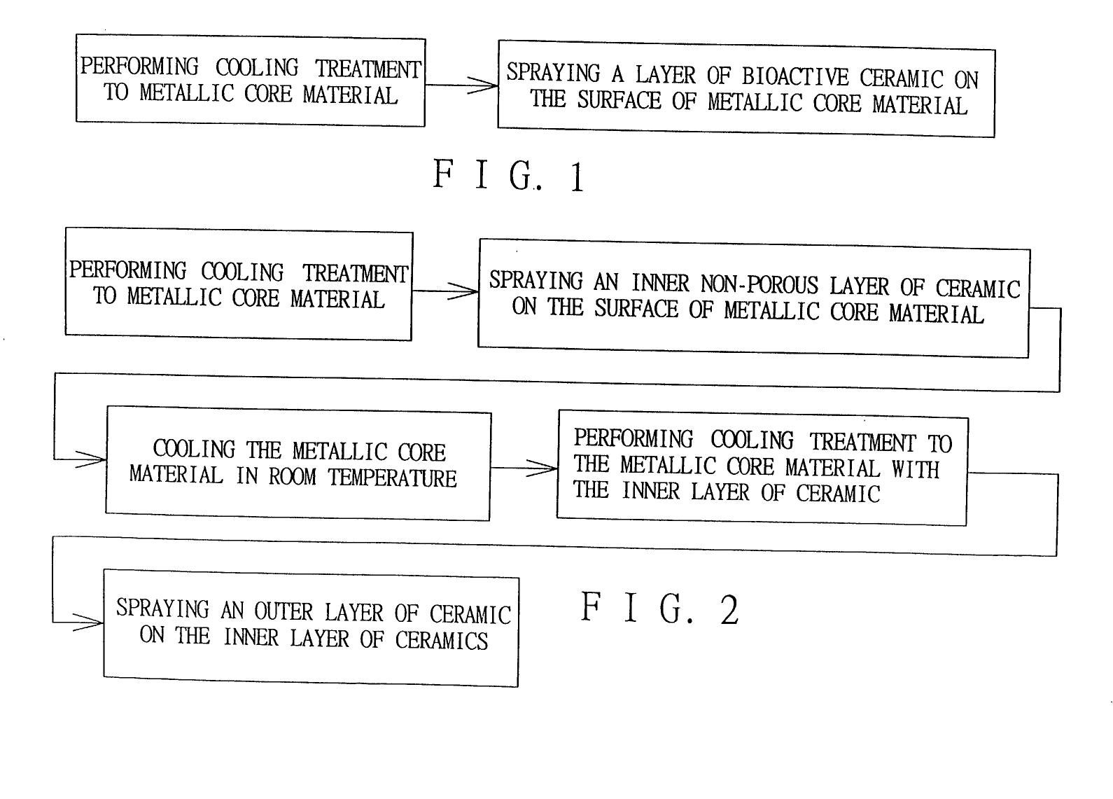

A first manufacturing method for surgical implants having a bioactive ceramics coating layer of the present invention, as shown in FIG. 1, includes plural steps. A first step is to treat metallic core materials by cooling them to a temperature within the range of −10° to 15° C., and preferably within −10° to 10° C., in a substantially dry environment, and then thermally spraying bioactive ceramics on the surface of the metallic core materials.

In the thermal spraying process, thermal expansion coefficient mismatch between the metallic core material and the ceramic coated layer cause minimal residual stress, resulting in strengthening of adhesion and bonding strength of the peripherally coated layer, because the metallic core material is cooled in advance.

Further, FIG. 2 shows a block diagram of the flow chart of a second manufacturing method of the present invention, wherein surgical implants having multi-layered structure are manufactured. In advance, the metallic base material is refrigerated to a low temperature, in the range of −10° to 15° C., and preferably within −10° to 10° C. Then, a layer of ceramic is coated on the surface of the metallic base material as an inner layer, i.e. an inner ceramic layer. Next, it is placed in a room temperature ambient to cool down naturally, and then refrigerated again to then be cooled to the same temperature as in the first step. Finally, bioactive ceramic material is sprayed with heat on the inner ceramic layer to make up an outer layer, i.e. a ceramic outer layer. Thus, the peripheral connection between the porous ceramic outer layer and the inner ceramic layer is reinforced further.

Metallic core materials include any metal generally used as artificial bones, joints, teeth, etc., not harmful to the human body, and having proper mechanical strength, for example, Co—Cr alloy, stainless steel, titanium, titanium alloy, or zirconium, etc.

Ceramic materials used as coating layers should not be harmful to the human body. For example, bioactive ceramic coating layers in the first manufacturing method and outer bioactive ceramic layers in a second manufacturing method may be (1) hydroxyapatite, (2) calcium phosphate, (3) Al₂O₃, (4) ZrO₂, (5) TiO₂, (6) SiO₂—Na₂O—CaO—P₂O, biodegradable glass, (7) porcelain powder, etc. Those materials can be used singly or mixed with two or more others. But, a ceramic layer containing hydroxyapatite is most preferable, as it has the best affinity to the body. Besides, the 16 inner ceramic layer in the second manufacturing method can be (1) glass-like Al₂O₃, TiO₂, Al₂O₃—SiO₂, etc. used as porcelain material, (2) Al₂O₃, (3) TiO₂, (4) ZrO₂, (5) SiO₂, etc.

The medium used in the cooling treatment of the invention does not have many limitations, only for keeping the coated surface clean and substantially dry, so common refrigerators can meet that need.

The thermal spraying for forming a plated layer in the invention is most preferably plasma spraying.

The manufacturing methods offered in the invention is not limited to metallic core materials with a ceramic layer formed by thermal spraying for surgical implants. The method can be applied to metallic core materials with a ceramic layer formed by thermal spraying that are applied to strengthen peripheral adhesion force therebetween, as is applied in a thermal barrier coating layer in turbine engines for aircraft.

In order to prove feasibility and functions of the present invention, experimental results of the first and the second manufacturing method will be shown below, in comparison with the conventional manufacturing methods.

(1) Embodiment of the first manufacturing method.

(1-1) A titanium alloy round rod of an inch diameter and 8 cm length was provided, and the cross-section of the rod was sand blasted. Then, the sand blasted rod was placed in a refrigerator at a temperature in the range of −10° to 15° C. for cooling. Then, hydroxyapatite (HA) was sprayed on the cross-section of the titanium rod by means of a plasma spraying process immediately after the cooling treatment. In a thermal spraying process, thermal expansion coefficient mismatch between the metallic core material and the ceramic layer coating causes minimal residual stress, resulting in strengthening of adhesion and bonding strength of he peripherally coated layer, because the metallic core material is pre-cooled prior to the thermal spraying process. Measurement of the thickness of the coated layer and its bonding strength was carried out according to ASTM C633-79 norm.

The resulting bonding strength had the value of 32.109±3.497MPa.

(1-2) The same sand blasting treatment to a titanium round rod of the same size was carried out, and then directly processed in a plasma spraying process, to the rod initially at room temperature, under the same condition of the plasma spraying process of the (1-1) trial, spraying hydroxyapatite (HA) on the cross-section of the rod. Measurement of the thickness and the bonding strength of the coated layer was carried out according to ASTM C633-79. The resulting bonding strength had the value of 28.551±3.215MPa.

(2) An embodiment of the second manufacturing method.

(2-1) A titanium alloy round rod of 1 inch diameter and 8 cm length was provided, and the cross-section of the rod was sand or grit blasted. Then, the rod was put in a refrigerator at a temperature in the range of ±10° to 15° C. A layer of non-porous ZrO₂, as an inner layer, was coated on the cross-section of the rod by means of plasma spraying process, with a typical thickness of 15-50 μm, immediately after the treatment of the rod. Then, the round rod with the ZrO₂ layer is naturally cooled down in a room temperature ambient exposed to a second cooling treatment in the refrigerator. Subsequently, hydroxyapatite (HA) is sprayed on the inner ZrO₂ layer as an outer layer, at the same conditions as above. In the thermal spraying process, thermal expansion coefficient mismatch between the metallic core material and the ceramic coated layer causes a minimal residual stress, resulting in strengthening of adhesion and bonding strength of the peripheral coated layer, because the metallic core material is pre-cooled prior to each thermal spraying process step. Measurement of the thickness and the bonding strength of the coated layer has been performed according to the ASTM C633-79 norm. Finally, the bonding strength had a value of 40.812±4.319MPa.

(2-2) The same sand blasting treatment of a titanium round rod of the same size, under the same conditions as that in trial (2-1) was carried out. Then, the plasma spraying process was applied to the sand blasted rod. The rod initially being at room temperature, when the cross-section of the rod is coated with a non-porous layer of ZrO₂ with a typical thickness of 15-50 μm. Then, the rod with the ZrO₂ layer was cooled down in a room temperature ambient. Next, a second plasma spraying process (under the same conditions as above) was carried out, (spraying a layer of hydroxyapatite on the ZrO₂ layer. Measurement of the thickness and bonding strength was performed according to ASTM C633-79 norm. The bonding strength was found to have a value of 36.209±3.017MPa.

The invention has the following advantages, among others, as can be understood in the aforesaid description.

1. The surgical implants made in accordance with the present invention can prevent metal ions from migrating out of he metallic core material, and compensate for the reduced peripheral adhesion between the outer ceramic layer and the inner layer.

2. The equipment use in the cooling treatment does not need to be special, so common refrigeration systems can meet the need. Thus, the method is easy and convenient to practice, and is cost effective.

3. The manufacturing method in the present invention can not only be used for surgical implants consisting of a metallic core material coated with ceramic layer by means of thermal spraying, but also used for other purposes where a metallic core material is coated with a ceramic layer, to strengthen the peripheral adhesion force therebetween.

What is being claimed is:

1. An improved manufacturing method for the strengthening of the bond strength of a one-layer bioactive ceramic or a two-layer bioactive ceramic having an inner ceramic layer which is coated onto a surgical implant including the steps of:
(a) artificially cooling a metallic core material to −10 to 10 degrees C; and
(b) spraying a bioactive ceramic layer on the surface of said metallic core material subsequent to cooling.

2. The improved manufacturing method for the strengthening of the bond strength of a bioactive ceramic layer coated onto the surgical implant as claimed in claim 1 wherein the step of cooling includes the step of refrigerating said metallic core material in an ambient atmosphere having a substantially dry environment.

3. The improved manufacturing method for the strengthening of the bond strength of bioactive ceramic layer coated onto the surgical implant as claimed in claim 1, wherein said metallic core material includes any metal used for artificial bones, joints, teeth, such as Co—Cr alloy, stainless steel, titanium, titanium alloy and zirconium, etc.

4. The improved manufacturing method for the strengthening of the bond strength of bioactive ceramic layer coated onto the surgical implant as claimed in claim 1, wherein the surface of said metallic core material of low temperature after freezing is thermal-sprayed with a ceramic layer containing at least calcium phosphate and hydroxyapatite (HA).

5. The improved manufacturing method for the strengthening of the bond strength of bioactive ceramic layer coated onto the surgical implant as claimed in claim 1, wherein said ceramic material thermal-sprayed on said metallic core material can be selected from those such as porcelain, Al₂O₃, ZrO₂, TiO₂, SiO₂—Na₂O—CaO—P₂O biodegradable glass, glass phase Al₂O₃ or TiO₂ and Al₂O₃—SiO₂.

6. An improved manufacturing method for the strengthening of the bond strength of two ceramic layers coated onto the surgical implant having an outer layer of bioactive ceramics including the steps of:
(a) initially cooling the metallic core material to −10 to 10 degrees C;
(b) thermal spraying an inner layer of ceramic on the surface of said metallic core material after said initial cooling;
(c) cooling the metallic core material coated with the inner layer of ceramic of step (b) to −10 degrees to 10 degrees C; and
(d) thermal spraying an outer layer of bioactive ceramic on the surface of the inner ceramic layer after said cooling of step (c).

7. The improved manufacturing method for the strengthening of the bond strength of two ceramic layers coated onto the surgical implant with the outer layer of bioactive ceramics as claimed in claim 6, wherein said freezing treatment is performed by and media supplying low temperature and a dry environment, such as common refrigerators used in homes.

8. The improved manufacturing method for the strengthening of the bond strength of two ceramic layers coated onto the surgical implant with the outer layer of bioactive ceramics as claimed in claim 6, wherein said metallic core material includes any metal used for artificial bones, joints, teeth, such as Co—Cr alloy, stainless steel, titanium alloy and zirconium, etc.

9. The improved manufacturing method for the strengthening of the bond strength of two ceramic layers coated onto the surgical implant with the outer layer of bioactive ceramics as claimed in claim 6, wherein said inner ceramic layer coated on the surface of said metallic core material is one or two mixed or more than two mixed ingredients of hydroxyapatite, calcium phosphate, porcelain powder, Al₂O₃, ZrO₂, TiO₂, SiO₂—Na₂O—CaO—P₂O biodegradable glass, glass phase Al₂O₃ or TiO₂ and Al₂O₃ or TiO₂ and Al₂O₃—SiO₂.

10. The improved manufacturing method for the strengthening of the bond strength of two ceramic layers coated onto the surgical implant with the outer layer of bioactive ceramics as claimed in claim 6, wherein said outer ceramic layer coated on said inner ceramic layer of low temperature after freezing treatment contains at least hydroxyapatite, and on said inner ceramic layer of low temperature after freezing treatment contains at least calcium phosphate, and hydroxyapatite (HA).

11. The improved manufacturing method for the strengthening of the bond strength of two ceramic layers coated onto the surgical implant with the outer layer of bioactive ceramics as claimed in claim 6, wherein said outer layer of sprayed ceramic on said surface of said inner ceramic layer is an ingredient selected from porcelain powder, Al₂O₃, ZrO₂, TiO₂, SiO₂—Na₂O—CaO—P₂O biodegradable glass, glass phase Al₂O₃ or TiO₂ and Al₂O₃—SiO₂.
